(19) United States
(12) Patent Application Publication
(10) Pub. No.: US 2023/0210733 A1
(43) Pub. Date: Jul. 6, 2023

(54) Oral Care Compositions and Methods of Use

(71) Applicant: Colgate-Palmolive Company, New York, NY (US)

(72) Inventors: Jean DENIS, Union, NJ (US); Robert D'AMBROGIO, Princeton, NJ (US); Guofeng XU, Plainsboro, NJ (US)

(21) Appl. No.: 18/091,571

(22) Filed: Dec. 30, 2022

Related U.S. Application Data
(60) Provisional application No. 63/295,168, filed on Dec. 30, 2021.

(51) Int. Cl. A61K 8/27; A61Q 11/00; A61K 8/21

(52) U.S. Cl. CPC A61K 8/27; A61Q 11/00; A61K 8/21; A61K 2800/262; A61K 2800/28; A61K 2800/92

(57) ABSTRACT

This disclosure relates to translucent oral care compositions comprising one or more source(s) of zinc source and/or a stannous source. The translucent oral care composition can comprise one or more zinc and/or stannous ion source(s) in amounts effective to provide at least 28% soluble zinc and/or stannous as a fraction of the total zinc and/or stannous ion concentration in the composition; an abrasive (e.g., silica), wherein the abrasive has a refractive index of approximately 1.45 as measured in a 4% silica, 90% sorbitol/water solution; and an orally acceptable vehicle. Methods of using and of making these compositions is also disclosed herein.

### FIELD OF THE INVENTION

This disclosure relates to translucent oral care compositions comprising one or more source(s) of a zinc source and/or a stannous source. In one aspect, the disclosure relates to translucent oral care compositions comprising one or more zinc ion source(s) and/or stannous ion source(s); wherein the zinc and/or stannous ion source(s) are in amounts effective to provide at least 28% soluble zinc and/or stannous as a fraction of the total zinc and/or stannous ion concentration in the composition; an abrasive (e.g., silica), wherein the abrasive has a refractive index of approximately 1.45 as measured in a 4% silica, 90% sorbitol/water solution; an orally acceptable vehicle. Methods of using and of making these compositions is also disclosed herein.

### BACKGROUND

Zinc is a known antimicrobial agent used in toothpaste compositions. Zinc is a known essential mineral for human health, and has been reported to help strengthen dental enamel and to promote cell repair.

Stannous ions, in particular stannous salts such as stannous fluoride, are also known anti-microbial agents and are used in various dentifrices as agents for preventing plaque. However, there are certain disadvantages to using stannous salts, such as instability, tendency to stain teeth, astringency, and unpleasant taste for users.

Unchecked bacterial growth in the oral cavity can lead to a number of adverse conditions. For example, gingivitis is an inflammation of the gums, and is one of the most common disorders of the oral cavity. It is ordinarily caused by bacterial accumulations on the surface of the teeth, which may be in the form of plaque. Gingivitis results in a number of unpleasant symptoms including inflamed gums that are painful or sensitive, halitosis, and bleeding from the gums while brushing or flossing. Other common disorders of the mouth include abscesses and cold sores, which also involve inflammation and are painful to those afflicted.

Soluble zinc salts, such as zinc citrate, have been used in dentifrice compositions, but have several disadvantages. Zinc ions in solution impart an unpleasant, astringent mouthfeel, so formulations that provide effective levels of zinc, and also have acceptable organoleptic properties, have been difficult to achieve. Moreover, free zinc ions may react with fluoride ions to produce zinc fluoride, which is insoluble and so reduces the availability of both the zinc and the fluoride. Finally, zinc ions can react with other dentifrice components, such as anionic surfactants (e.g., sodium lauryl sulfate), interfering with foaming and cleaning and composition stability.

Soluble metal ions, such as stannous and zinc, may also react unfavorably with polymeric rheological modifiers, such as modified celluloses (e.g., carboxymethyl cellulose) and gums (e.g., xanthan gum or carrageenan gum). Such compounds often considered to be incompatible with divalent metal ions.

Traditionally, the emphasis in developing metal-ion based oral care compositions has been to maximize the concentration of soluble zinc and soluble stannous ions, because it was believed that only soluble forms of these ions contribute to antibacterial efficacy. However, from a marketing or aesthetic perspective, the appearance of a dentifrice is very important. In the past, toothpastes were always white and completely opaque. Over the last few decades, transparent or translucent toothpastes have become very common. Consumers are very attracted to transparent toothpastes, which are commonly made in colors such as red, green and blue. The degree of transparency can vary, but often takes considerable effort to control, as the color and transparency together can depend on many factors, including the coloring agents and their concentrations, the refractive index of the composition, the opacity of other ingredients (such as silicas and polymers), and the water content of the composition.

Translucent toothpastes containing relatively high amounts of stannous and/or zinc are difficult to formulate given that the presence of metals can make the composition cloudy. For example, it is typically assumed that to have a translucent toothpaste with zinc and/or stannous that the concentration or fraction of insoluble metal ion needs to be relatively low, while the fraction or concentration of soluble metal ion needs to be relatively high. Consequently, this requirement can adversely affect the efficacy of the resulting toothpaste despite its transparent aesthetic.

Accordingly, there is a need in the marketplace for a translucent toothpaste that also has the efficacy of stannous and zinc containing compositions that can comprise relatively high amounts of insoluble metal salts.

### BRIEF SUMMARY

The following description of embodiment(s) is merely exemplary in nature and is in no way intended to limit the invention, its application, or uses.

As used throughout, ranges are used as shorthand for describing each and every value that is within the range. Any value within the range can be selected as the terminus of the range. In addition, all references cited herein are hereby incorporated by referenced in their entireties. In the event of a conflict in a definition in the present disclosure and that of a cited reference, the present disclosure controls.

Without being bound by theory, the disclosure provides for translucent oral care compositions that can comprise relatively little soluble metal ion concentration (e.g., no less than 25% soluble metal ion concentration) but are still clear or translucent in appearance and retain the efficacy (e.g., antimicrobial efficacy) normally associated with oral care compositions that contain stannous and zinc metal actives. In one aspect, the translucent oral care compositions comprise one or more zinc and/or stannous ion source(s), wherein the zinc and/or stannous ion source(s) provides at least 25% soluble zinc and/or stannous in the composition (as a fraction of the total zinc and/or stannous); and wherein the composition comprises an abrasive (e.g., silica), wherein the abrasive has a refractive index of between 1-2 (e.g., about 1.40 to about 1.50; e.g., about 1.45) as measured in a 4% silica, 90% sorbitol/water solution.

In one aspect the disclosure provides a translucent oral care composition
(Composition 1.0) comprising:
   a. One or more zinc ion source(s) and/or stannous ion source(s); wherein the zinc and/or stannous ion source(s) are in amounts effective to provide at least 28% soluble zinc and/or stannous as a fraction of the total zinc and/or stannous ion concentration in the composition;
   b. An abrasive (e.g., silica), wherein the abrasive has a refractive index of approximately 1-2 (e.g., about 1.45) as measured in a 4% silica, 90% sorbitol/water solution; and
   c. An orally acceptable vehicle.
      For example, the invention contemplates any of the following compositions (unless otherwise indicated, values are given as percentage of the overall weight of the composition):
1.1 Composition 1.0, wherein the composition comprises one or more zinc ion sources, and wherein the one or more sources of zinc ion source(s) comprises a zinc salt selected from the group consisting of: zinc citrate, zinc oxide, zinc phosphate, zinc lactate, zinc sulfate, zinc silicate and combinations thereof.
1.2 Composition 1.0 or 1.1, wherein the composition comprises a source of zinc ion, and wherein the zinc ion source comprises zinc oxide (e.g., from 0.25-1.25% by wt. of the total composition) (e.g., about 0.5%) (e.g., about 1%).
1.3 Composition 1.0 or 1.1, wherein the composition comprises a source of zinc ion, and wherein the zinc ion source comprises zinc citrate (e.g., from 0.25-1.5% by wt. of the total composition) (e.g., about 0.5%) (e.g., about 1%) (e.g., about 1.35%).
1.4 Composition 1.0 or 1.1, wherein the composition comprises one or more sources of zinc ion, and wherein the one or more sources of zinc ion comprises zinc oxide (e.g., from 0.25%-1.25% by wt. of the total composition) and zinc citrate (e.g., from 0.25%-1.5% by wt. of the total composition).
1.5 Composition 1.0 or 1.1, wherein the composition comprises a source of zinc ion, and wherein the zinc ion source comprises zinc phosphate (e.g., wherein the zinc phosphate is a preformed salt of zinc phosphate) (e.g., zinc phosphate hydrate) (e.g., from 0.5%-4% by wt. of the total composition) (e.g., from 0.5-2% by wt. of the total composition) (e.g., about 1.0 wt. % of zinc phosphate).
1.6 Any of the preceding compositions further comprising a polyphosphate.
1.7 Any of the preceding compositions, wherein the composition comprises a source of zinc, and wherein the zinc source comprises zinc oxide and zinc citrate, and wherein the ratio of the amount of zinc oxide (e.g., wt. %) to zinc citrate (e.g., wt. %) is from 1.5:1 to 4.5:1 (e.g., 2:1, 2.5:1, 3:1, 3.5:1, or 4:1).
1.8 Any of the preceding compositions, wherein the composition comprises a source of zinc, and wherein the zinc source comprises zinc oxide and zinc citrate, and wherein the zinc citrate is in an amount of from 0.25 to 1 wt. % (e.g., 0.5 wt. %) and zinc oxide may be present in an amount of from 0.75 to 1.25 wt. % (e.g., 1.0 wt. %) based on the weight of the oral care composition.
1.9 Any of the preceding compositions wherein the composition comprises a source of zinc, wherein the source of zinc comprises zinc citrate, and wherein the zinc citrate is about 0.5 wt. % (e.g., zinc citrate trihydrate).
1.10 Any of the preceding compositions wherein the composition comprises a source of zinc, wherein the source of zinc comprises zinc oxide, and wherein the zinc oxide is about 1.0 wt. %.
1.11 Any of the preceding compositions wherein the composition comprises a source of zinc, wherein the source of zinc comprises zinc citrate and zinc oxide, and where the zinc citrate is about 0.5 wt. % and the zinc oxide is about 1.0 wt. %.
1.12 Any of the preceding compositions wherein the composition comprises a source of zinc, wherein the source of zinc comprises zinc citrate and zinc lactate, and wherein the ratio of the amount of zinc oxide (e.g., wt. %) to zinc lactate (e.g., wt. %) is from 1.2:1 to 4.5:1 (e.g., 1.25:1, 2:1, 2.5:1, 3:1, 3.5:1, or 4:1).
1.13 Any of the preceding compositions, wherein the composition comprises a stannous ion source.
1.14 A composition according to the preceding, wherein the stannous ion source is selected from the group consisting of: stannous fluoride, other stannous halides (e.g., stannous chloride dihydrate), stannous pyrophosphate, organic stannous carboxylate salts (e.g., stannous formate, acetate, lactate, tartrate, oxalate, malonate and citrate), stannous ethylene glyoxide, and combinations thereof
1.15 Any of the preceding compositions, wherein the stannous ion source is stannous fluoride (e.g., from 0.1-2% by wt. of the composition).
1.16 Any of the preceding compositions, wherein the stannous fluoride is present in an amount of 0.1 wt. % to 2 wt. % (0.1 wt. %-0.6 wt. %) (e.g., about 0.454 wt. %) of the total composition weight.
1.17 Any of the preceding compositions wherein the stannous fluoride is in an amount from 50 to 25,000 ppm (e.g., 750-7000 ppm, e.g., 1000-5000 ppm, e.g., about 4500 ppm, e.g., about 4540 ppm).
1.18 Any of the preceding compositions, wherein the composition comprises stannous fluoride and stannous pyrophosphate.
1.19 Any of the preceding compositions, wherein the composition comprises a zinc ion source but not a stannous ion source.
1.20 Any of the preceding compositions, wherein the composition comprises a stannous ion source but does not contain a zinc ion source.
1.21 Any of Composition 1.0-1.20, wherein the composition comprises both a zinc ion source and a stannous ion source.
1.22 Any of the preceding compositions, wherein the composition comprises a copolymer.
1.23 The composition of 1.22, wherein the copolymer is a PVM/MA copolymer.
1.24 The composition of 1.23, wherein the PVM/MA copolymer comprises a 1:4 to 4:1 copolymer of maleic anhydride or acid with a further polymerizable ethylenically unsaturated monomer; for example 1:4 to 4:1, e.g., about 1:1.
1.25 Any of the preceding compositions, wherein the further polymerizable ethylenically unsaturated monomer comprises methyl vinyl ether (methoxyethylene).
1.26 Any of the preceding compositions, wherein the PVM/MA copolymer comprises a copolymer of methyl vinyl ether/maleic anhydride, wherein the anhydride is hydrolyzed following copolymerization to provide the corresponding acid.
1.27 Any of the preceding compositions, wherein the PVM/MA copolymer comprises a GANTREZ® polymer (e.g., GANTREZ® S-97 polymer)
1.28 Any of the preceding compositions wherein the pH is between 6.5 and 9.5. e.g., about 7.0 or about 8.0.
1.29 Any of the preceding compositions further comprising a fluoride ion source.
1.30 The composition of 1.30, wherein the fluoride ion source is selected from the group consisting of stannous fluoride, sodium fluoride, potassium fluoride, sodium monofluorophosphate, sodium fluorosilicate, ammonium fluorosilicate, amine fluoride, ammonium fluoride, and combinations thereof
1.31 The composition of 1.30, wherein the fluoride ion source is sodium fluoride and/or sodium monofluorophosphate.
1.32 Any of the preceding compositions comprising a polyphosphate, wherein the polyphosphate is sodium tripolyphosphate (STPP).
1.33 The composition of 1.32, wherein the sodium tripolyphosphate is from 0.5-5.0 wt. % (e.g., about 3.0 wt. %).
1.34 Any of the preceding compositions further comprising an effective amount of one or more alkali phosphate salts, e.g., sodium, potassium or calcium salts, e.g., selected from alkali dibasic phosphate and alkali pyrophosphate salts, e.g., alkali phosphate salts selected from sodium phosphate dibasic, potassium phosphate dibasic, dicalcium phosphate dihydrate, calcium pyrophosphate, tetrasodium pyrophosphate, tetrapotassium pyrophosphate, disodium hydrogenorthophosphate, monosodium phosphate, pentapotassium triphosphate and mixtures of any of two or more of these, e.g., in an amount of 1-20%, e.g., 2-8%, e.g., ca. 5%>, by weight of the composition.
1.35 The composition of 1.34, wherein the alkali phosphate salt comprises tetrasodium pyrophosphate from 0.5-5.0 wt. % (e.g., about 3.0 wt. %).
1.36 Any of the preceding compositions further comprising an abrasive or particulate (e.g., silica).
1.37 Any of the preceding compositions wherein the silica is synthetic amorphous silica. (e.g., 1%-25% by wt.) (e.g., 8%-25% by wt.) (e.g., about 12% by wt.)
1.38 Any of the preceding composition wherein the silica abrasives are silica gels or precipitated amorphous silicas, e.g., silicas having an average particle size ranging from 2.5 microns to 12 microns.
1.39 Any of the preceding compositions further comprising a small particle silica having a median particle size (d50) of 1-5 microns (e.g., 3-4 microns) (e.g., about 5 wt. % Sorbosil AC43 from Ineos Silicas, Warrington, United Kingdom).
1.40 Any of the preceding compositions wherein 20-30 wt. % of the total silica in the composition is small particle silica (e.g., having a median particle size (d50) of 3-4 microns) and wherein the small particle silica is about 5 wt. % of the oral care composition.
1.41 Any of the preceding compositions comprising silica wherein the silica is used as a thickening agent, e.g., particle silica.
1.42 Any of the preceding compositions, wherein the orally acceptable vehicle comprises one or more of water, a thickener, a buffer, a humectant, a surfactant, a sweetener, a pigment, a dye, an anti-caries agent, an anti-bacterial, a whitening agent, a desensitizing agent, a vitamin, a preservative, and mixtures thereof.
1.43 Any of the preceding compositions further comprising an anionic surfactant, wherein the anionic surfactant is in an amount of from 0.5-5% by wt., e.g., 1-2% by weight, selected from water-soluble salts of higher fatty acid monoglyceride monosulfates, (e.g., sodium N-methyl N-cocoyl taurate), sodium cocomo-glyceride sulfate; higher alkyl sulfates, (e.g., sodium lauryl sulfate); higher alkyl-ether sulfates (e.g., of formula CH₃(CH₂)ₘCH₂(OCH₂CH₂)ₙOSO₃X, wherein m is 6-16, e.g., 10, n is 1-6, e.g., 2, 3 or 4, and X is Na) or (e.g., sodium laureth-2 sulfate (CH₃(CH₂)₁₀CH₂(OCH₂CH₂)₂OSO₃Na); higher alkyl aryl sulfonates (e.g., sodium dodecyl benzene sulfonate, sodium lauryl benzene sulfonate); higher alkyl sulfoacetates (e.g., sodium lauryl sulfoacetate; dodecyl sodium sulfoacetate), higher fatty acid esters of 1,2 dihydroxy propane sulfonate, sulfocolaurate (e.g., N-2-ethyl laurate potassium sulfoacetamide) and sodium lauryl sarcosinate, and mixtures thereof
1.44 Any of the preceding compositions, wherein the anionic surfactant is sodium lauryl sulfate.
1.45 Any of the preceding compositions further comprising glycerin.
1.46 Any of the preceding compositions comprising polymer films.
1.47 Any of the preceding compositions comprising a flavoring agent, fragrance and/or coloring.
1.48 The composition of 1.47, wherein the flavoring agent is sodium saccharin, sucralose, or a mixture thereof.
1.49 Any of the preceding compositions, wherein the composition comprises one or more thickening agent(s) selected from the group consisting of carboxyvinyl polymers, carrageenan, xanthan, hydroxyethyl cellulose and water-soluble salts of cellulose ethers (e.g., sodium carboxymethyl cellulose and sodium carboxymethyl hydroxyethyl cellulose) and combinations thereof.
1.50 Any of the preceding compositions, wherein the composition comprises sodium carboxymethyl cellulose (e.g., from 0.1 wt. %-2.5 wt. %) (e.g., about 0.2% by wt.).
1.51 Any of the preceding compositions comprising from 5%-40%, e.g., 10%-35%, e.g., about 10, about 12%, about 15%, about 18%, about 20%, about 25%, about 30%, and about 35% water.
1.52 Any of the preceding compositions comprising an additional antibacterial agent selected from halogenated diphenyl ether (e.g. triclosan), herbal extracts and essential oils (e.g., rosemary extract, tea extract, magnolia extract, thymol, menthol, eucalyptol, geraniol, carvacrol, citral, hinokitol, catechol, methyl salicylate, epigallocatechin gallate, epigallocatechin, gallic acid, miswak extract, sea-buckthorn extract), bisguanide antiseptics (e.g., chlorhexidine, alexidine or octenidine), quaternary ammonium compounds (e.g., cetylpyridinium chloride (CPC), benzalkonium chloride, tetradecylpyridinium chloride (TPC), N-tetradecyl-4-ethylpyridinium chloride (TDEPC)), phenolic antiseptics, hexetidine, octenidine, sanguinarine, povidone iodine, delmopinol, salifluor, metal ions (e.g., zinc salts, for example, Zinc Chloride, Zinc Lactate, Zinc Sulfate, stannous salts, copper salts, iron salts), sanguinarine, propolis and oxygenating agents (e.g., hydrogen peroxide, buffered sodium peroxyborate or peroxycarbonate), phthalic acid and its salts, monoperthalic acid and its salts and esters, ascorbyl stearate, oleoyl sarcosine, alkyl sulfate, dioctyl sulfosuccinate, salicylanilide, domiphen bromide, delmopinol, octapinol and other piperidino derivatives, nicin preparations, chlorite salts; and mixtures of any of the foregoing.
1.53 Any of the preceding compositions comprising an antioxidant, e.g., selected from the group consisting of Co-enzyme Q10, PQQ, Vitamin C, Vitamin E, Vitamin A, BHT, anethole-dithiothione, and mixtures thereof.
1.54 Any of the preceding compositions further comprising an agent that interferes with or prevents bacterial attachment, e.g., ELA or chitosan.
1.55 Any of the preceding compositions further a buffer system; (e.g., wherein the buffer comprises trisodium citrate and citric acid).
1.56 Any of the preceding compositions, wherein the composition comprises an aqueous buffer system, for example, wherein the buffer system comprises an organic acid and an alkali metal salt thereof, e.g., wherein the organic acid is citric acid and the salt is a mono-, di- and/or tri-alkali metal citrate salt, e.g., mono-, di- and/or tri-lithium, sodium, potassium, or cesium citrate salt, and citric acid. For example, where the composition comprises 1-10% by weight organic acid salt and 0.1-5% by weight organic acid.
1.57 The composition of 1.56, wherein the buffer system comprises a citrate buffer, wherein the citrate buffer comprises tri-sodium citrate and citric acid (e.g., 1 to 10% by weight of the composition), for example, wherein the molar ratio of mono-, di- and/or tri-sodium citrate and citric acid is 1.5 to 5, (e.g., 2 to 4).
1.58 Any of the preceding compositions comprising:
   a. zinc oxide (e.g., about 1.0%)
   b. zinc citrate (e.g., zinc citrate trihydrate) (e.g., about 0.5% zinc citrate)
   c. stannous fluoride (e.g., about 0.45% stannous fluoride);
   d. An abrasive (e.g., silica), wherein the abrasive has a refractive index of approximately 1.45 as measured in a 4% silica, 90% sorbitol/water solution;
   e. An orally acceptable carrier; and
   f. Wherein the amounts of zinc oxide and zinc citrate are effective to provide at least 28% soluble zinc ion concentration as a fraction of the total concentration in the composition.
1.59 Any of Composition 1.0-1.58, wherein the composition comprises:
   a. zinc phosphate (e.g., about 1.0% zinc phosphate)
   b. stannous fluoride (e.g., about 0.45% stannous fluoride);
   c. An abrasive (e.g., silica), wherein the abrasive has a refractive index of approximately 1.45 as measured in a 4% silica, 90% sorbitol/water solution;
   d. An orally acceptable carrier;
   e. wherein the amount of zinc phosphate and stannous fluoride is effective to provide at least 28% soluble metal ion concentration (e.g., the total amount of soluble zinc and stannous ion concentration) as a fraction of the total concentration in the composition.
1.60 Any of Composition 1.0-1.58, wherein the composition comprises:
   a. zinc phosphate (about 1.0% zinc phosphate)
   b. stannous fluoride (e.g., about 0.45% stannous fluoride); and
   c. An abrasive (e.g., silica), wherein the abrasive has a refractive index of approximately 1.45 as measured in a 4% silica, 90% sorbitol/water solution;
   d. wherein the amount of zinc phosphate and stannous fluoride is effective to provide at least 28% soluble metal ion concentration (e.g., relative to the total amount of soluble zinc and stannous ion concentration) as a fraction of the total concentration in the composition; and
   e. wherein the amount of water is more than 10% by wt. of the composition (e.g., from 10%-30% by wt.) (e.g., about 15% by wt.) (e.g., about 16% by wt.) (e.g., about 17% by wt.) (e.g., about 18% by wt.) (e.g., about 19% by wt.) (e.g., about 20% by wt.); and
   f. an orally acceptable carrier.
1.61 Any of Composition 1.0-1.58, wherein the composition comprises:
   a. Zinc oxide and/or zinc citrate
   b. A fluoride source (e.g., sodium fluoride);
   c. an abrasive (e.g., silica), wherein the abrasive has a refractive index of approximately 1.45 as measured in a 4% silica, 90% sorbitol/water solution;
   d. an orally acceptable carrier; and
   e. wherein the amounts of zinc oxide and zinc citrate are effective to provide at least 28% soluble zinc ion concentration as a fraction of the total concentration in the composition.
1.62 Any of Composition 1.0-1.58, wherein the composition comprises:
   a. zinc citrate; and
   b. An abrasive (e.g., silica), wherein the abrasive has a refractive index of approximately 1.45 as measured in a 4% silica, 90% sorbitol/water solution;
   c. Wherein the amount of zinc citrate are effective to provide at least 28% soluble zinc ion concentration as a fraction of the total concentration in the composition;
   d. An orally acceptable carrier; and
   e. an abrasive (e.g., silica), wherein the abrasive has a refractive index of approximately 1.45 as measured in a 4% silica, 90% sorbitol/water solution;
1.63 Any of the preceding compositions further comprising microcrystalline cellulose/sodium carboxymethylcellulose, e.g., in an amount of from 0.1-5%, e.g., 0.5-2%, e.g., about 1% by wt.
1.64 Any of the preceding compositions further comprising polyvinylpyrrolidone (PVP) in an amount of from 0.5-3 wt. %, e.g., about 1.25 wt. %.
1.65 Any of the preceding compositions effective upon application to the oral cavity, e.g., by rinsing, optionally in conjunction with brushing, to (i) reduce or inhibit formation of dental caries, (ii) reduce, repair or inhibit pre-carious lesions of the enamel, e.g., as detected by quantitative light-induced fluorescence (QLF) or electrical caries measurement (ECM), (iii) reduce or inhibit demineralization and promote remineralization of the teeth, (iv) reduce hypersensitivity of the teeth, (v) reduce or inhibit malodor, (vi) promote healing of sores or cuts in the mouth, (vii) reduce levels of acid producing bacteria, (ix) inhibit microbial biofilm formation in the oral cavity, (x) raise and/or maintain plaque pH at levels of at least pH 5.5 following sugar challenge, (xi) reduce plaque accumulation, (xii) treat, relieve or reduce dry mouth, (xiii) clean the teeth and oral cavity (xiv) reduce erosion, (xv) prevents stains and/or whiten teeth, (xvi) immunize the teeth against cariogenic bacteria; and/or (xvii) promote systemic health, including cardiovascular health, e.g., by reducing potential for systemic infection via the oral tissues.
1.66 Any of the preceding oral compositions, wherein the oral composition may be any of the following oral compositions selected from the group consisting of: a toothpaste or a dentifrice, a mouthwash or a mouth rinse, a topical oral gel (e.g., an oral gel meant for office or professional use), a chewing gum, a dental tray application, mouth spray, foam, tablet, powder, a non-abrasive gel, a mousse, a denture cleanser, a coated or impregnated immediate or delayed release oral adhesive strip or patch, and a coated or impregnated oral wipe or swab.
1.67 Any of the preceding compositions, where the only source of zinc ion consists of zinc oxide and/or zinc citrate.
1.68 Any of the preceding compositions, where the only source of zinc ion is zinc oxide.
1.69 Any of the preceding compositions, where the only source of stannous is stannous fluoride.
1.70 A composition obtained or obtainable by combining the ingredients as set forth in any of the preceding compositions.
1.71 Any of the preceding oral compositions, wherein the composition is incorporated into a toothpaste.
1.72 Any of the preceding compositions, further comprising an amino acid source, wherein the amino acid source comprises an amino acid selected from the group consisting of arginine, L-arginine, cysteine, leucine, isoleucine, lysine, L-lysine, alanine, asparagine, aspartate, phenylalanine, glutamate, glutamic acid, threonine, glutamine, tryptophan, glycine, valine, proline, serine, tyrosine, histidine, and combinations thereof
1.73 Any of the preceding compositions, wherein the amino acid has the L-configuration (e.g., L-arginine).
1.74 Any of the preceding compositions, wherein the amino acid source comprises a basic amino acid.
1.75 Any of the preceding compositions, wherein the amino acid source comprises an amino acid selected from the group consisting of arginine, lysine, glycine and combinations thereof.
1.76 Any of the preceding compositions, wherein the amino acid source comprises arginine.
1.77 Any of Composition 1.0-1.58, wherein the composition comprises:
   a. Zinc oxide and/or zinc citrate
   b. sodium fluoride;
   c. an abrasive (e.g., silica), wherein the abrasive has a refractive index of approximately 1.45 as measured in a 4% silica, 90% sorbitol/water solution;
   d. wherein the amounts of zinc oxide and zinc citrate are effective to provide at least 28% soluble zinc ion concentration as a fraction of the total concentration in the composition;
   e. an orally acceptable carrier; and
   f. arginine (e.g., from 0.5-6% by wt. relative to the total composition).
1.78 Any of the preceding compositions, wherein the composition comprises:
   a. zinc phosphate (about 1.0% zinc phosphate)
   b. stannous fluoride (e.g., about 0.45% stannous fluoride); and
   c. An abrasive (e.g., silica), wherein the abrasive has a refractive index of approximately 1.45 as measured in a 4% silica, 90% sorbitol/water solution;
   d. wherein the amount of zinc phosphate and stannous fluoride is effective to provide at least 28% soluble metal ion concentration (e.g., the total amount of soluble zinc and stannous ion concentration) as a fraction of the total concentration in the composition; and
   e. an orally acceptable carrier;
   wherein the amount of water is more than 10% by wt. of the composition (e.g., from 10%-30% by wt.) (e.g., about 15% by wt.) (e.g., about 16% by wt.) (e.g., about 17% by wt.) (e.g., about 18% by wt.) (e.g., about 19% by wt.) (e.g., about 20% by wt.); and
   f. Arginine (e.g., from 0.5-6% by wt. relative to the total composition).
1.79 Any of the preceding compositions, wherein the composition comprises an effective amount of a taurate surfactant, wherein the taurate surfactant is represented by Formula (1):

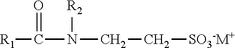

wherein R₁ is a saturated or unsaturated, straight or branched alkyl chain with 6 to 18 C atoms R₂ is H or methyl, and M⁺ is H, sodium, or potassium (e.g., sodium methyl cocoyl taurate).
1.80 The preceding composition, wherein the R₁ is a saturated or unsaturated, straight or branched alkyl chain with 8 to 14 C atoms.
1.81 Composition 1.84 or 1.85, wherein the taurate surfactant comprises one or more surfactant selected from the group consisting of: potassium cocoyl taurate, potassium methyl cocoyl taurate, sodium caproyl methyl taurate, sodium cocoyl taurate, sodium lauroyl taurate, sodium methyl cocoyl taurate (SMCT), sodium methyl lauroyl taurate, sodium methyl myristoyl taurate, sodium methyl oleoyl taurate, sodium methyl palmitoyl taurate, sodium methyl stearoyl taurate, and combinations thereof.
1.82 Any of the preceding compositions, wherein the taurate surfactant comprises one or more surfactant selected from the group consisting of: sodium lauroyl methyl taurate (or sodium methyl lauroyl taurate), sodium methyl cocoyl taurate (SMCT), and combinations thereof.
1.83 Any of the preceding compositions, wherein the taurate surfactant comprises sodium methyl cocoyl taurate (e.g., 1%-5% by wt. of sodium methyl cocoyl taurate) (e.g., about 2% by wt. sodium methyl cocoyl taurate).
1.84 Any of the preceding compositions, wherein the taurate surfactant is present in an amount of from 0.25% to 5%, e.g., from 0.4% to 3%, e.g., from 0.4% to 2.75%, e.g., from 0.4% to 2.5%, e.g., from 0.5% to 3%, e.g., from 0.8% to 3%, e.g., from 1% to 3%, e.g., from 1.2% to 2.7%, e.g., from 1.5% to 3%, e.g., from 2% to 3%, e.g., from 1% to 2.8%, e.g., from 1% to 2.7%, e.g., from 1% to 2.5%, e.g., from 1.5% to 2.8%, e.g., from 1.5% to 2.5%, e.g., from 1.8% to 3%, e.g., from 1.8% to 2.8%, e.g., from 1.8% to 2.7%, e.g., from 1.8% to 2.5%, e.g., about 2% by weight of the composition.
1.85 Any of the preceding compositions, wherein the composition comprises a silica abrasive having a N₂ BET surface area of less than 50 m²/g and an Einlehner hardness of from 4 to 11.
1.86 Any of the preceding compositions, wherein the silica abrasive (e.g., Sylodent VP5) has the following physical properties:

TABLE 1

| | Sylodent VP5 |
|---|---|
| N₂ BET surface area (m²/g) | <50 |
| Oil absorption (cc/100 g) | 80-100 |
| Mean particle size (μm) | 9-13 |
| d10 (μm) | 2.74 |
| Brass Einlehner hardness | 6-9 |

1.87 The preceding compositions, wherein the silica abrasive has a refractive index of approximately 1.45 as measured in a 4% silica, 90% sorbitol/water solution.
1.88 Any of the preceding compositions, wherein the composition has L*a*b color values of 1-50 for L (e.g., 5 to 30 or 10 to 20), and/or −0.1 to 0.1 for a, and/or −0.25 to 0.25 for b.
1.89 The preceding composition, wherein the composition has L*a*b color values of about 12 for L, about 0 for a and about 0.2 for b.
1.90 Any of the preceding compositions, wherein the composition does not comprise any white pigment (e.g., does not comprise titanium dioxide).
1.91 Any preceding composition, wherein the composition comprises a zinc ion source(s) in an amount of from 0.05 to 10% by weight, relative to the weight of the oral care composition, for example, from 0.1 to 8% by weight, or from 0.5 to 5% by weight, or from 0.5 to 4% by weight, or from 1 to 4%, or from 1 to 3% by weight, or from 2 to 3% by weight, or about 1% or about 2%, or about 2.25% or about 2.5%, by weight.
1.92 Any preceding composition, wherein the composition comprises a stannous ion source in an amount of from 0.05 to 10% by weight, relative to the weight of the oral care composition, for example, from 0.1 to 8% by weight, or from 0.5 to 5% by weight, or from 0.5 to 4% by weight, or from 1 to 4%, or from 1 to 3% by weight, or from 2 to 3% by weight, or about 1% or about 2%, or about 2.25% or about 2.5%, by weight.
1.93 Any preceding composition, wherein the composition comprises sodium citrate, e.g., mono-, di- and/or tri-sodium citrate.
1.94 The preceding composition, wherein the composition comprises trisodium citrate (e.g., in an amount effective to provide a clear or translucent oral care composition).
1.95 The preceding composition, wherein the composition comprises trisodium citrate in an amount from 2%-7% by wt. of the total composition (e.g., about 2% by wt.).
1.96 The preceding composition, wherein the composition comprises trisodium citrate in an amount from 2.5%-6.5% by wt. of the total composition (e.g., from 2%-4% by wt.) (e.g., about 3% by wt.) (e.g., about 3.5% by wt.) (e.g., about 6.5% by wt.) (e.g., about 4% by wt.) (e.g., about 5% by wt.).
1.97 Any of the preceding compositions, wherein the amount of soluble metal ion in the composition (e.g., the total amount of soluble zinc or stannous relative to the total amount of metal ion in the composition) is from 28%-95% (e.g., from 28%-50%) (e.g., from 28%-45%) (e.g., from 28%-40%) (e.g., from 28%-35%) (e.g., about 28%) (e.g., about 30%) (e.g., about 35%).
1.98 Any of the preceding compositions comprising nitric acid or a water-soluble nitrate salt (e.g., potassium nitrate).
1.99 The preceding composition, wherein the water-soluble nitrate salt is selected from an alkali or alkaline earth metal nitrate, or zinc nitrate, silver nitrate, or ammonium nitrate.
1.100 The preceding composition, wherein the water-soluble nitrate salt is an alkali metal nitrate salt or an alkaline earth metal nitrate salt.
1.101 The preceding composition, wherein the nitrate salt is selected from lithium nitrate, sodium nitrate, potassium nitrate, magnesium nitrate, and calcium nitrate.
1.102 The preceding composition, wherein the nitrate salt is potassium nitrate.
1.103 Any preceding composition, wherein the oral care composition is free or substantially free of sodium lauryl sulfate.
1.104 Any of the preceding compositions, wherein the oral care composition is in the form of a translucent gel.
1.105 Any of the preceding compositions wherein the composition has a light transmittance of at least 4% measured in a sample cube having an approximately 25 mm-path length, e.g., at least 7%, e.g., at least 8%, e.g., at least 9%, e.g., at least 10%, e.g., from 10%-30%, e.g., from 11%-25%.
1.106 Any of the preceding compositions, wherein the composition has a turbidity of less than 500 NTU measured in a sample cube having an approximately 25 mm-path length, e.g., less than 400 NTU, or less than 350 NTU, or 75-500 NTU, or from 80-350 NTU; or from 80-200 NTU; or from 80-150 NTU.

A composition for use as set forth in any of the preceding compositions, e.g., any of Composition 1.0 et seq.

In another embodiment, the invention encompasses a method to improve oral health comprising applying an effective amount of the oral composition of any of the embodiments (e.g., any of Compositions 1.0 et seq) set forth above to the oral cavity of a subject in need thereof, e.g., a method to
i. reduce or inhibit formation of dental caries,
ii. reduce levels of acid producing bacteria,
iii. inhibit microbial bio film formation in the oral cavity,
iv. reduce plaque accumulation,
v. immunize (or protect) the teeth against cariogenic bacteria and their effects, and/or
vi. clean the teeth and oral cavity.

In another aspect, the present disclosure provides a method for producing a translucent oral care composition (Composition 2), e.g., an oral care composition (e.g., any of Composition 1.0 et seq), wherein the method comprises combining one or more zinc ion source(s) and/or stannous ion source(s) in an orally acceptable carrier (e.g., wherein the zinc and/or stannous ion source(s) are in amounts effective to provide at least 28% soluble zinc and/or stannous as a fraction of the total zinc and/or stannous ion concentration in the composition); and an abrasive (e.g., silica), wherein the abrasive has a refractive index of approximately 1-2 (e.g., about 1.40 to about 1.50; e.g., 1.45) as measured in a 4% silica, 90% sorbitol/water solution; and sodium citrate (e.g., trisodium citrate) in an amount from 2%-7% by wt. of the total composition.

The invention further comprises the use of sodium bicarbonate, sodium methyl cocoyl taurate (tauranol), MIT, and benzyl alcohol and combinations thereof in the manufacture of a Composition of the Invention, e.g., for use in any of the indications set forth in the above method of Composition 1.0, et seq.

In a further aspect, the invention contemplates a method of decreasing mitochondrial respiration (e.g., oxygen consumption rate) and/or glycolysis (e.g., measured by extracellular acidification rate) in an oral biofilm of a subject in need thereof, wherein the method comprises administering any of Composition 1.0 et seq to the oral cavity of the subject.

In a further aspect, the invention contemplates a method for increasing: a) antibacterial efficacy; and/or b) optical transmission; of an aqueous oral care composition, the composition comprising one or more zinc ion source(s) and/or stannous ion source(s), and an abrasive (e.g., silica), wherein the abrasive has a refractive index of approximately 1.45 as measured in a 4% silica, 90% sorbitol/water solution; the method comprising formulating the composition to include a zinc ion and/or stannous ion solubilizing agent; e.g. wherein the solubilizing agent comprises citrate ion; e.g. wherein the solubilizing agent comprises trisodium citrate; e.g., in an amount from 2%-7% by wt. of the total composition. In some embodiments, the method comprises formulating the composition in accordance with any of the Compositions 1 and 1.1-1.106.

### DETAILED DESCRIPTION

As used herein, the term “dentifrice” means paste, gel, or liquid formulations unless otherwise specified. The dentifrice composition can be in any desired form such as deep striped, surface striped, multi-layered, having the gel surrounding the paste, or any combination thereof. Alternatively, the oral composition may be dual phase dispensed from a separated compartment dispenser.

As used herein, an “oral care composition” refers to a composition for which the intended use includes oral care, oral hygiene, and/or oral appearance, or for which the intended method of use comprises administration to the oral cavity, and refers to compositions that are palatable and safe for topical administration to the oral cavity, and for providing a benefit to the teeth and/or oral cavity. The term “oral care composition” thus specifically excludes compositions which are highly toxic, unpalatable, or otherwise unsuitable for administration to the oral cavity. In some embodiments, an oral care composition is not intentionally swallowed, but is rather retained in the oral cavity for a time sufficient to affect the intended utility. The oral care compositions as disclosed herein may be used in nonhuman mammals such as companion animals (e.g., dogs and cats), as well as by humans. In some embodiments, the oral care compositions as disclosed herein are used by humans. Oral care compositions include, for example, dentifrice and mouthwash. In some embodiments, the disclosure provides mouthwash formulations.

As used herein, “orally acceptable” refers to a material that is safe and palatable at the relevant concentrations for use in an oral care formulation, such as a mouthwash or dentifrice.

As used herein, “orally acceptable carrier” refers to any vehicle useful in formulating the oral care compositions disclosed herein. The orally acceptable carrier is not harmful to a mammal in amounts disclosed herein when retained in the mouth, without swallowing, for a period sufficient to permit effective contact with a dental surface as required herein. In general, the orally acceptable carrier is not harmful even if unintentionally swallowed. Suitable orally acceptable carriers include, for example, one or more of the following: water, a thickener, a buffer, a humectant, a surfactant, an abrasive, a sweetener, a flavorant, a pigment, a dye, an anti-caries agent, an anti-bacterial, a whitening agent, a desensitizing agent, a vitamin, a preservative, an enzyme, and mixtures thereof.

As used herein throughout, the terms “soluble” and “solubility” refer to aqueous solubility (i.e., the solubility of the described species in water). As used herein, the term “soluble” refers to a compound having a solubility product constant (K_{SP}) in water of greater than or equal to 1×10⁻¹⁰ (at 20° C.). As used herein, the term “insoluble” refers to a compound having a solubility product constant (K_{SP}) in water of less than 1×10⁻¹⁰ (at 20° C.).

Insoluble zinc compounds include, but are not limited to, zinc oxide, zinc phosphate, zinc pyrophosphate, zinc silicate, zinc oleate, zinc hydroxide, zinc carbonate, zinc peroxide and zinc sulfide. By way of comparison, soluble zinc compounds include zinc citrate, zinc chloride, zinc lactate, zinc nitrate, zinc acetate, zinc glycinate and zinc sulfate.

Insoluble stannous compounds include, but are not limited to, stannous phosphate (i.e., stannous orthophosphate), stannous pyrophosphate, stannous oxide, stannous sulfate, stannous peroxide, and stannous hydroxide. By way of comparison, soluble stannous compounds include stannous fluoride, stannous chloride, stannous nitrate and stannous sulfate.

As used herein throughout, the term “zinc ion and/or stannous ion solubilizing agent” refers to a compound that functions in the formulation to increase the solubility of one or both of zinc ions and stannous ions. Examples of such solubilizing agents include citrate salts, for example trisodium citrate; e.g., in an amount from 2%-7% by wt. of the total composition.

### Fluoride Ion Source

The oral care compositions of the disclosure, e.g., any of Composition 1.0 et seq., may further include one or more fluoride ion sources, e.g., soluble fluoride salts. A wide variety of fluoride ion-yielding materials can be employed as sources of soluble fluoride in the present compositions. Examples of suitable fluoride ion-yielding materials are found in U.S. Pat. No. 3,535,421, to Briner et al.; U.S. Pat. No. 4,885,155, to Parran, Jr. et al. and U.S. Pat. No. 3,678,154, to Widder et al., each of which are incorporated herein by reference. Representative fluoride ion sources used with the present invention (e.g., Composition 1.0 et seq.) include, but are not limited to, stannous fluoride, sodium fluoride, potassium fluoride, sodium monofluorophosphate, sodium fluorosilicate, ammonium fluorosilicate, amine fluoride, ammonium fluoride, and combinations thereof. In certain embodiments the fluoride ion source includes stannous fluoride, sodium fluoride, sodium monofluorophosphate as well as mixtures thereof. Where the formulation comprises calcium salts, the fluoride salts are preferably salts wherein the fluoride is covalently bound to another atom, e.g., as in sodium monofluorophosphate, rather than merely ionically bound, e.g., as in sodium fluoride.

### Surfactants

The oral care compositions of the disclosure, e.g., any of Composition 1.0 et seq., may contain anionic surfactants, for example, water-soluble salts of higher fatty acid monoglyceride monosulfates, such as the sodium salt of the monosulfated monoglyceride of hydrogenated coconut oil fatty acids such as sodium N-methyl N-cocoyl taurate, sodium cocomo-glyceride sulfate; higher alkyl sulfates, such as sodium lauryl sulfate; higher alkyl-ether sulfates, e.g., of formula CH₃(CH₂)ₘCH₂(OCH₂CH₂)ₙOSO₃X, wherein m is 6-16, e.g., 10, n is 1-6, e.g., 2, 3 or 4, and X is Na or, for example sodium laureth-2 sulfate (CH₃(CH₂)₁₀CH₂(OCH₂CH₂)₂OSO₃Na); higher alkyl aryl sulfonates such as sodium dodecyl benzene sulfonate (sodium lauryl benzene sulfonate); higher alkyl sulfoacetates, such as sodium lauryl sulfoacetate (dodecyl sodium sulfoacetate), higher fatty acid esters of 1,2 dihydroxy propane sulfonate, sulfocolaurate (N-2-ethyl laurate potassium sulfoacetamide) and sodium lauryl sarcosinate. By “higher alkyl” is meant, e.g., C₆₋₃o alkyl. In particular embodiments, the anionic surfactant (where present) is selected from sodium lauryl sulfate and sodium ether lauryl sulfate. When present, the anionic surfactant is present in an amount which is effective, e.g., >0.001% by weight of the formulation, but not at a concentration which would be irritating to the oral tissue, e.g., 1%, and optimal concentrations depend on the particular formulation and the particular surfactant. In one embodiment, the anionic surfactant is present at from 0.03% to 5% by weight, e.g., about 1.75% by wt.

In another embodiment, cationic surfactants useful in the present invention can be broadly defined as derivatives of aliphatic quaternary ammonium compounds having one long alkyl chain containing 8 to 18 carbon atoms such as lauryl trimethylammonium chloride, cetyl pyridinium chloride, cetyl trimethylammonium bromide, di-isobutylphenoxyethyldimethylbenzylammonium chloride, coconut alkyltrimethylammonium nitrite, cetyl pyridinium fluoride, and mixtures thereof. Illustrative cationic surfactants are the quaternary ammonium fluorides described in U.S. Pat. No. 3,535,421, to Briner et al., herein incorporated by reference. Certain cationic surfactants can also act as germicides in the compositions.

Illustrative nonionic surfactants of the disclosure, e.g., any of Composition 1.0, et seq., that can be used in the compositions of the disclosure can be broadly defined as compounds produced by the condensation of alkylene oxide groups (hydrophilic in nature) with an organic hydrophobic compound which may be aliphatic or alkylaromatic in nature. Examples of suitable nonionic surfactants include, but are not limited to, the Pluronics, polyethylene oxide condensates of alkyl phenols, products derived from the condensation of ethylene oxide with the reaction product of propylene oxide and ethylene diamine, ethylene oxide condensates of aliphatic alcohols, long chain tertiary amine oxides, long chain tertiary phosphine oxides, long chain dialkyl sulfoxides and mixtures of such materials. In a particular embodiment, the composition of the invention comprises a nonionic surfactant selected from polaxamers (e.g., polaxamer 407), polysorbates (e.g., polysorbate 20), polyoxyl hydrogenated castor oils (e.g., polyoxyl 40 hydrogenated castor oil), and mixtures thereof.

Illustrative amphoteric surfactants of Composition 1.0, et seq., that can be used in the compositions of the invention include betaines (such as cocamidopropylbetaine), derivatives of aliphatic secondary and tertiary amines in which the aliphatic radical can be a straight or branched chain and wherein one of the aliphatic substituents contains about 8-18 carbon atoms and one contains an anionic water-solubilizing group (such as carboxylate, sulfonate, sulfate, phosphate or phosphonate), and mixtures of such materials.

The surfactant or mixtures of compatible surfactants can be present in the compositions of the present invention in 0.1% to 5%, in another embodiment 0.3% to 3% and in another embodiment 0.5% to 2% by weight of the total composition.

### Flavoring Agents

The oral care compositions of the disclosure, e.g., any of Composition 1.0 et seq., may also include a flavoring agent. Flavoring agents which are used in the practice of the present invention include, but are not limited to, essential oils and various flavoring aldehydes, esters, alcohols, and similar materials, as well as sweeteners such as sodium saccharin. Examples of the essential oils include oils of spearmint, peppermint, wintergreen, sassafras, clove, sage, eucalyptus, marjoram, cinnamon, lemon, lime, grapefruit, and orange. Also useful are such chemicals as menthol, carvone, and anethole. Certain embodiments employ the oils of peppermint and spearmint.

The flavoring agent is incorporated in the oral composition at a concentration of 0.01 to 1% by weight.

### pH Adjusting Agents

In some embodiments, the compositions of the present disclosure, e.g., any of Composition 1.0 et seq, contain a buffering agent. Examples of buffering agents include anhydrous carbonates such as sodium carbonate, sesquicarbonates, bicarbonates such as sodium bicarbonate, silicates, bisulfates, phosphates (e.g., monopotassium phosphate, monosodium phosphate, disodium phosphate, dipotassium phosphate, tribasic sodium phosphate, sodium tripolyphosphate, pentapotassium tripolyphosphate, phosphoric acid), citrates (e.g. citric acid, trisodium citrate dehydrate), pyrophosphates (sodium and potassium salts, e.g., tetrapotassium pyrophosphate) and combinations thereof. The amount of buffering agent is sufficient to provide a pH of about 5 to about 9, preferable about 6 to about 8, and more preferable about 7, when the composition is dissolved in water, a mouthrinse base, or a toothpaste base. Typical amounts of buffering agent are about 5% to about 35%, in one embodiment about 10% to about 30%, in another embodiment about 15% to about 25%, by weight of the total composition.

### Chelating and Anti-Calculus Agents

The oral care compositions of the disclosure, e.g., any of Composition 1.0 et seq., also may include one or more chelating agents able to complex calcium found in the cell walls of the bacteria. Binding of this calcium weakens the bacterial cell wall and augments bacterial lysis.

Another group of agents suitable for use as chelating or anti-calculus agents in the present invention are the soluble pyrophosphates. The pyrophosphate salts used in the present compositions can be any of the alkali metal pyrophosphate salts. In certain embodiments, salts include tetra alkali metal pyrophosphate, dialkali metal diacid pyrophosphate, trialkali metal monoacid pyrophosphate and mixtures thereof, wherein the alkali metals are sodium or potassium. The salts are useful in both their hydrated and unhydrated forms. An effective amount of pyrophosphate salt useful in the present composition is generally enough to provide at least 0.1 wt. % pyrophosphate ions, e.g., 0.1 to 3 wt. %, e.g., 0.1 to 2 wt. %, e.g., 0.1 to 1 wt. %, e.g., 0.2 to 0.5 wt. %. The pyrophosphates also contribute to preservation of the compositions by lowering water activity.

Suitable anticalculus agents for the compositions of the disclosure (e.g., any of Composition 1.0 et seq) include without limitation phosphates and polyphosphates (for example pyrophosphates), polyaminopropanesulfonic acid (AMPS), hexametaphosphate salts, zinc citrate trihydrate, polypeptides, polyolefin sulfonates, polyolefin phosphates, diphosphonates. In particular embodiments, the invention includes alkali phosphate salts, i.e., salts of alkali metal hydroxides or alkaline earth hydroxides, for example, sodium, potassium or calcium salts. “Phosphate” as used herein encompasses orally acceptable mono- and polyphosphates, for example, P₁₋₆ phosphates, for example monomeric phosphates such as monobasic, dibasic or tribasic phosphate; dimeric phosphates such as pyrophosphates; and multimeric phosphates, e.g., sodium hexametaphosphate. In particular examples, the selected phosphate is selected from alkali dibasic phosphate and alkali pyrophosphate salts, e.g., selected from sodium phosphate dibasic, potassium phosphate dibasic, dicalcium phosphate dihydrate, calcium pyrophosphate, tetrasodium pyrophosphate, tetrapotassium pyrophosphate, sodium tripolyphosphate, and mixtures of any of two or more of these. In a particular embodiment, for example the compositions comprise a mixture of tetrasodium pyrophosphate (Na₄P₂O₇), calcium pyrophosphate (Ca₂P₂O₇), and sodium phosphate dibasic (Na₂HPO₄), e.g., in amounts of ca. 3-4% of the sodium phosphate dibasic and ca. 0.2-1% of each of the pyrophosphates. In another embodiment, the compositions comprise a mixture of tetrasodium pyrophosphate (TSPP) and sodium tripolyphosphate (STPP)(Na₅P₃O₁₀), e.g., in proportions of TSPP at about 1-2% and STPP at about 7% to about 10%. Such phosphates are provided in an amount effective to reduce erosion of the enamel, to aid in cleaning the teeth, and/or to reduce tartar buildup on the teeth, for example in an amount of 2-20%, e.g., ca. 5-15%, by weight of the composition.

### Polymers

The oral care compositions of the disclosure, e.g., any of Composition 1.0 et seq., also optionally include one or more polymers, such as polyethylene glycols, polyvinyl methyl ether maleic acid copolymers, polysaccharides (e.g., cellulose derivatives, for example carboxymethyl cellulose, or polysaccharide gums, for example xanthan gum or carrageenan gum). Acidic polymers, for example polyacrylate gels, may be provided in the form of their free acids or partially or fully neutralized water soluble alkali metal (e.g., potassium and sodium) or ammonium salts. Certain embodiments include 1:4 to 4:1 copolymers of maleic anhydride or acid with another polymerizable ethylenically unsaturated monomer, for example, methyl vinyl ether (methoxyethylene) having a molecular weight (M.W.) of about 30,000 to about 1,000,000. These copolymers are available for example as Gantrez AN 139 (M.W. 500,000), AN 1 19 (M.W. 250,000) and S-97 Pharmaceutical Grade (M.W. 70,000), of GAF Chemicals Corporation.

Other operative polymers include those such as the 1:1 copolymers of maleic anhydride with ethyl acrylate, hydroxyethyl methacrylate, N-vinyl-2-pyrollidone, or ethylene, the latter being available for example as Monsanto EMA No. 1 103, M.W. 10,000 and EMA Grade 61, and 1:1 copolymers of acrylic acid with methyl or hydroxyethyl methacrylate, methyl or ethyl acrylate, isobutyl vinyl ether or N-vinyl-2-pyrrolidone.

The N-vinyl-2-pyrrolidione is also commonly known as polyvinylpyrrolidone or “PVP”. PVP refers to a polymer containing vinylpyrrolidone (also referred to as N-vinylpyrrnlidone and N-vinyl-2-pyrrolidinone) as a monomeric unit. The monomeric unit consists of a polar imide group, four non-polar methylene groups and a non-polar methane group. The polymers include soluble and insoluble homopolymeric PVPs. Copolymers containing PVP include vinylpyrrolidone/vinyl acetate (also known as Copolyvidone, Copolyvidonum or VP-VAc) and vinyl pyrrolidone/dimethylamino-ethylmethacrylate. Soluble PVP polymers among those useful herein are known in the art, including Povidone, Polyvidone, Polyvidonum, poly(N-vinyl-2-pyrrolidinone), poly (N-vinylbutyrolactam), poly(1-vinyl-2-pyrrolidone) and poly [1-(2-oxo-1 pyrrolidinyl)ethylene]. These PVP polymers are not substantially cross-linked. In some embodiments the polymer comprises an insoluble cross-linked homopolymer. Such polymers include crosslinked PVP (often referred to as cPVP, polyvinylpolypyrrolidone, or cross-povidone).

Suitable generally, are polymerized olefinically or ethylenically unsaturated carboxylic acids containing an activated carbon-to-carbon olefinic double bond and at least one carboxyl group, that is, an acid containing an olefinic double bond which readily functions in polymerization because of its presence in the monomer molecule either in the alpha-beta position with respect to a carboxyl group or as part of a terminal methylene grouping. Illustrative of such acids are acrylic, methacrylic, ethacrylic, alpha-chloroacrylic, crotonic, beta-acryloxy propionic, sorbic, alpha-chlorsorbic, cinnamic, beta-styrylacrylic, muconic, itaconic, citraconic, mesaconic, glutaconic, aconitic, alpha-phenylacrylic, 2-benzyl acrylic, 2-cyclohexylacrylic, angelic, umbellic, fumaric, maleic acids and anhydrides. Other different olefinic monomers copolymerizable with such carboxylic monomers include vinylacetate, vinyl chloride, dimethyl maleate and the like. Copolymers contain sufficient carboxylic salt groups for water-solubility.

A further class of polymeric agents includes a composition containing homopolymers of substituted acrylamides and/or homopolymers of unsaturated sulfonic acids and salts thereof, in particular where polymers are based on unsaturated sulfonic acids selected from acrylamidoalykane sulfonic acids such as 2-acrylamide 2 methylpropane sulfonic acid having a molecular weight of about 1,000 to about 2,000,000, described in U.S. Pat. No. 4,842,847, Jun. 27, 1989 to Zahid, incorporated herein by reference.

In preparing oral care compositions, it is sometimes necessary to add some thickening material to provide a desirable consistency or to stabilize or enhance the performance of the formulation. In certain embodiments, the thickening agents are carboxyvinyl polymers, carrageenan, xanthan, hydroxyethyl cellulose and water soluble salts of cellulose ethers such as sodium carboxymethyl cellulose and sodium carboxymethyl hydroxyethyl cellulose. Natural gums such as karaya, gum arabic, and gum tragacanth can also be incorporated. Colloidal magnesium aluminum silicate or finely divided silica can be used as component of the thickening composition to further improve the composition's texture. In certain embodiments, thickening agents in an amount of about 0.5% to about 5.0% by weight of the total composition are used.

In some embodiments, microcrystalline cellulose (MCC) can be used (e.g., carboxymethyl cellulose with sodium carboxymethyl cellulose). An example of a source of MCC is Avicel® (FMC Corporation), which contains MCC in combination with sodium carboxymethyl cellulose (NaCMC). Both Avicel®. RC-591 (MCC containing 8.3 to 13.8 weight % NaCMC) and Avicel®. CL-611 (MCC containing 11.3 to 18.8 weight % NaCMC) may be used in certain aspects. In certain embodiments, the ratio of microcrystalline cellulose to cellulose ether thickening agent is from 1:1 to 1:3 by weight; or from 1:1.5 to 1:2.75 by weight. In any of the above embodiments comprising sodium carboxymethylcellulose, microcrystalline cellulose may be used in combination with NaCMC. In certain such embodiments, the MCC/sodium carboxymethylcellulose may be present in an amount of from 0.5 to 1.5 weight % based on the total weight of the composition.

### Abrasives

In certain embodiments the compositions of the disclosure may comprise additional calcium-containing abrasives, for example calcium phosphate abrasive, e.g., tricalcium phosphate (Ca₃(PO₄)₂), hydroxyapatite (Ca₁₀(PO₄)₆(OH)₂), or dicalcium phosphate dihydrate (CaHPO₄.2H₂O, also sometimes referred to herein as DiCal) or calcium pyrophosphate, and/or silica abrasives, sodium metaphosphate, potassium metaphosphate, aluminum silicate, calcined alumina, bentonite or other siliceous materials, or combinations thereof. Any silica suitable for oral care compositions may be used, such as precipitated silicas or silica gels. For example synthetic amorphous silica. Silica may also be available as a thickening agent, e.g., particle silica. For example, the silica can also be small particle silica (e.g., Sorbosil AC43 from PQ Corporation, Warrington, United Kingdom). However the additional abrasives are preferably not present in a type or amount so as to increase the RDA of the dentifrice to levels which could damage sensitive teeth, e.g., greater than 130.

Useful silica abrasive materials for preparing the oral compositions of the present invention, e.g., any of Compositions 1.0 et seq, may be obtained from Davison Chemical Division of W. R. Grace & Co. (Baltimore, Md., USA) under the tradename Sylodent VP5, as described in United States Patent Application 2012/0100193 (the contents of which are incorporated herein by reference). The physical properties of Sylodent VP5 are shown in Table 1.

TABLE 1

| | Sylodent VP5 |
|---|---|
| N₂ BET surface area (m²/g) | <50 |
| Oil absorption (cc/100 g) | 80-100 |
| Mean particle size (μm) | 9-13 |
| d10 (μm) | 2.74 |
| Brass Einlehner hardness | 6-9 |

The use of Sylodent VP5 in oral care compositions can impart a superior cleaning ability, e.g., a high PCR value, and at the same time, reduces damage to hard dental surfaces, e.g., a low RDA, as shown in United States Patent Application 2012/0100193.

### Water

Water is present in the oral compositions of the invention. Water, employed in the preparation of commercial oral compositions should be deionized and free of organic impurities. Water commonly makes up the balance of the compositions and includes 5% to 45%, e.g., 10% to 20%, e.g., 25-35%, by weight of the oral compositions. This amount of water includes the free water which is added plus that amount which is introduced with other materials such as with sorbitol or silica or any components of the invention. The Karl Fischer method is a one measure of calculating free water.

### Humectants

Within certain embodiments of the oral compositions, it is also desirable to incorporate a humectant to reduce evaporation and also contribute towards preservation by lowering water activity. Certain humectants can also impart desirable sweetness or flavor to the compositions. The humectant, on a pure humectant basis, generally includes 15% to 70% in one embodiment or 30% to 65% in another embodiment by weight of the composition.

Suitable humectants include edible polyhydric alcohols such as glycerin, sorbitol, xylitol, propylene glycol as well as other polyols and mixtures of these humectants. Mixtures of glycerin and sorbitol may be used in certain embodiments as the humectant component of the compositions herein.

### Amino Acids

In some aspects, Compositions 1.0 et seq can comprise a basic amino acid. The basic amino acids which can be used in the compositions and methods of the invention include not only naturally occurring basic amino acids, such as arginine, lysine, and histidine, but also any basic amino acids having a carboxyl group and an amino group in the molecule, which are water-soluble and provide an aqueous solution with a pH of 7 or greater.

For example, basic amino acids include, but are not limited to, arginine, lysine, serine, citrulline, ornithine, creatine, histidine, diaminobutanoic acid, diaminoproprionic acid, salts thereof or combinations thereof. In a particular embodiment, the basic amino acids are selected from arginine, citrulline, and ornithine. In certain embodiments, the basic amino acid is arginine, for example, L-arginine, or a salt thereof.

In another aspect, the compositions of the invention (e.g., Compositions 1.0 et seq) can further comprise one or more neutral amino acid, which can include, but is not limited to, one or more neutral amino acids selected from the group consisting of alanine, aminobutyrate, asparagine, cysteine, cystine, glutamine, glycine, hydroxyproline, isoleucine, leucine, methionine, phenylalanine, proline, serine, taurine, threonine, tryptophan, tyrosine, valine, and combinations thereof.

The compositions and methods according to the invention (e.g., Composition 1.0 et seq) can be incorporated into oral compositions for the care of the mouth and teeth such as dentifrices, toothpastes, transparent pastes, gels, mouth rinses, sprays and chewing gum.

The toothpaste making process involves sufficient mixing for a homogenous product. In some embodiments, the later part of the process (after the gel phase and once silica is added) is performed under vacuum, for example at least about −26 mmHg, to remove entrapped air bubbles that could contribute to finished product opacity.

As used throughout, ranges are used as shorthand for describing each and every value that is within the range. Any value within the range can be selected as the terminus of the range. In addition, all references cited herein are hereby incorporated by reference in their entireties. In the event of a conflict in a definition in the present disclosure and that of a cited reference, the present disclosure controls. It is understood that when formulations are described, they may be described in terms of their ingredients, as is common in the art, notwithstanding that these ingredients may react with one another in the actual formulation as it is made, stored and used, and such products are intended to be covered by the formulations described.

The following examples further describe and demonstrate illustrative embodiments within the scope of the present invention. The examples are given solely for illustration and are not to be construed as limitations of this invention as many variations are possible without departing from the spirit and scope thereof. Various modifications of the invention in addition to those shown and described herein should be apparent to those skilled in the art and are intended to fall within the appended claims.

### EXAMPLES

### Example 1

The following are representative formulas of the present disclosure (ingredients listed as percentages by wt. of the total formulations) (*Note: tables reflect total Zn, Sn, total insoluble metals per analytical evaluation):

TABLE 1

| Ingredients. | A | B | C | D |
|---|---|---|---|---|
| Polymers | 4% | 4% | 4% | 4% |
| Sweetener | 2% | 2% | 2% | 2% |
| and Flavor | | | | |
| Sod. Fluoride | 0.243% | 0.243% | 0.243% | 0.32% |
| Humectant | 51.83% | 51.83% | 51.83% | 47.60% |
| Water | q. s. | q. s. | q. s. | q. s. |
| Silica VP5 | 20% | 20% | 20% | 20% |
| Thickener | 5% | 5.5% | 5% | 6% |
| Anionic | 1.5% | 1.5% | 1.5% | 1.5% |
| Surfactant | | | | |
| Zwitterionic | 1.25% | 1.25% | 1.25% | 1.25% |
| Surfactant | | | | |
| (30% | | | | |
| solution) | | | | |
| Trisodium | 3% | — | 3% | 6.5% |
| Citrate | | | | |
| Citric Acid | — | — | — | — |
| Zn Oxide | 0.5% | 0.5% | 1% | 1% |
| Zn Citrate | 1.35% | 1.35% | 0.5% | 0.5% |
| Arginine | 1.3% | 1.3% | 1.3% | 1.3% |
| Total Zinc | 0.82% | 0.82% | 0.96% | 0.96% |
| Total | 0.48% | 0.68% | 0.7% | 0.6% |
| insoluble zinc | | | | |
| Appearance | Clear | Opaque | Opaque | Clear |
| % of soluble | 47% | 17% | 27% | 37.5% |
| zinc relative | | | | |
| to total zinc | | | | |

TABLE 2

| Ingredients | H | J |
|---|---|---|
| Polymers | 3.5% | 4% |
| Sweetener and | 2% | 2% |
| Flavor | | |
| Sod. Fluoride | 0.243% | 0.32% |
| Humectant | 55.87% | 54.1 |
| Water | 7% | 8% |
| Silica (e.g., | 20% | 20% |
| Sylodent VP5) | | |
| Thickener | 5% | 6% |
| Anionic | 1.5% | 1.5% |
| Surfactant | | |
| Zwitterionic | 1.25% | 1.25% |
| Surfactant | | |
| Trisodium Citrate | 2% | — |
| Citric Acid | 0.1% | — |
| Zn Oxide | 0.5% | 1% |
| Zn Citrate | — | 0.5% |
| Arginine | — | 1.3% |
| Total Zinc | 0.43% | 0.96% |
| Total insoluble | 0.31% | 0.84% |
| zinc | | |
| Appearance | Clear | Opaque |
| % of soluble zinc | 28% | 12.5% |
| relative to total | | |
| zinc | | |

Further representative formulas containing both zinc and stannous ion sources:

TABLE 3

| Ingredients. | N | O | P | Q |
|---|---|---|---|---|
| Polymers | 4% | 4% | 4% | 4% |
| Flavor and | 2% | 2% | 2% | 2% |
| Sweetener | | | | |
| Stannous | 0.454% | 0.454% | 0.454% | 0.454% |
| Fluoride | | | | |
| Humectant | 52.27% | 54.77% | 54.57% | 50% |
| Water | q.s. | q.s. | q.s. | q.s. |
| Silica | 20% | 20% | 20% | 20% |
| (Sylodent | | | | |
| VP5) | | | | |
| Thickener | 5% | 5% | 5% | 5.25% |
| Anionic | 1.5% | 1.5% | 1.5% | 1.5% |
| Surfactant | | | | |
| Zwitterionic | 1.25% | 1.25% | 1.25% | 1.25% |
| Surfactant | | | | |
| Trisodium | 3% | 3.5% | — | 4% |
| Citrate | | | | |
| Citric Acid | — | 0.1% | — | — |
| Alkali | 1% | 1.2% | 1.2% | 1.8% |
| Phosphate | | | | |
| Salt | | | | |
| Zn Oxide | — | — | — | — |
| Zinc Citrate | 1% | — | — | — |
| Zinc | — | 1% | 1% | 1% |
| Phosphate | | | | |
| Potassium | 0.5% | — | — | — |
| Nitrate | | | | |
| Arginine | — | — | — | — |
| Total Zinc | 0.32 | 0.45 | 0.45 | 0.44 |
| Total Tin | 0.35 | 0.35 | 0.35 | 0.35 |
| Total | 0.14% | 0.23% | 0.33% | 0.18% |
| Insoluble Zinc | | | | |
| Total | 0.01 | 0.04 | 0.14 | 0.07 |
| Insoluble Tin | | | | |
| Total Metal | 0.67 | 0.8 | 0.8 | 0.79 |
| Total | 0.15% | 0.27% | 0.47 | 0.25 |
| Insoluble | | | | |
| Metal | | | | |
| Appearance | Clear | Clear | Opaque | Clear |
| % of soluble | 77.4% | 66% | 41% | 68% |
| metal (zinc | | | | |
| and stannous) | | | | |
| relative to | | | | |
| total metal | | | | |

Insoluble zinc (and stannous) were determined for each formula by subtracting soluble metal analytical results from total zinc (and stannous) analytical results.

### Example 2

An in-vitro Plaque Glycolysis model was utilized to compare antibacterial efficacy of toothpaste formulations containing significantly different levels of soluble metal. The study details are as follows.

Plaque glycolysis Model: An in-vitro adaptation of a published Plaque Glycolysis Model (Donald J. White, et. al., Journal of Clinical Dentistry, #6 Special Issue, Pp 69-78, 1995) was used to indirectly measure biofilm health. Briefly, the method quantifies the glycolytic effects of toothpaste formulas on treated in vitro biofilm pool of both anaerobic and aerobic bacteria. The efficacy of each toothpaste formula is based on biofilm pH change. A lower average pH change indicates reduction of viable bacteria and greater antibacterial performance of the respective test toothpaste. Finally, in these studies, an untreated cell is used as the negative control.

In one test (Table 4 below), two common formulations containing the same total zinc metal but different levels of soluble zinc are compared (Formulas A & B from Example 1 above). It can be seen that sample A with 3% trisodium citrate and 47% of the zinc in a soluble state provides significantly greater reduction in viable bacteria compared to the common Formula B. Formula B, as the main point of difference to formula A, does not contain trisodium citrate and contains only 17% of the zinc in a soluble state.

TABLE 4
Plaque Glycolysis Study—Average pH Change with Treatment (Test 1)

| | | Statistical |
|---|---|---|
| Sample | Avg pH Change | Comparison* |
| Untreated** | 2.519 +/− 0.095 | A |
| Toothpaste B | 0.882 +/− 0.033 | B |
| Toothpaste A | 0.529 +/− 0.048 | C |

*Means that don't share common letter = Sign. Diff @95% CI, Tukey method, N = 3 per cell.
**Negative Control, untreated biofilm

In a second plaque glycolysis test, two other common formulations containing the same total zinc metal but different levels of soluble zinc are compared (Formulas C & D from Example 1). The results are shown in Table 5 below. Again, it was observed that the toothpaste (D) with 37.5% of the zinc in soluble form provides greater reduction in viable bacteria compared to the toothpaste C with only 27% of soluble zinc relative to total zinc. Again, the difference in performance is statistically significant and indicates that more soluble metal typically provides improved antibacterial performance in an otherwise equivalent formulation.

TABLE 5
Plaque Glycolysis Study: Average pH Change with Treatment (Test 2)

| | | Statistical |
|---|---|---|
| Sample | Avg pH Change | Comparison* |
| Untreated** | 2.622 +/− 0.078 | A |
| Toothpaste C | 0.712 +/− 0.024 | B |
| Toothpaste D | 0.603 +/− 0.037 | C |

*Means that don't share common letter = Sign. Diff @95% CI, Tukey method, N = 3 per cell.
**Negative Control, untreated biofilm.

In a third plaque glycolysis test, two formulations are compared that have a common base with the same target levels of total stannous and zinc metals but demonstrate different soluble metal levels by means of analytical evaluations (Formulas O & P from Example 1). The results are shown in Table 6 below. The higher soluble metal in Formula O is due to inclusion of 3.5% trisodium citrate which Formula P does not contain. Again, the difference in performance is statistically significant and indicates that more soluble metal typically provides improved antibacterial performance in an otherwise equivalent formulation.

TABLE 6
Plaque Glycolysis Study—Average pH Change with Treatment (Test 3)

| | | Statistical |
|---|---|---|
| Sample | Avg pH Change | Comparison* |
| Untreated** | 2.743 +/− 0.092 | A |
| Toothpaste P | 0.916 +/− 0.067 | B |
| Toothpaste O | 0.579 +/− 0.052 | C |

*Means that don't share common letter = Sign. Diff @95% CI, Tukey method, N = 3 per cell.
**Negative Control, untreated biofilm.

### Example 3

Determination of Gel Transparency

Determination of gel transparency was determined by subjective visual measurements, wherein a ribbon of toothpaste is squeezed onto a sheet of white paper containing typed text. The samples are rated on a rating scale of 1 to 10, where a 10 is given if the text can be read perfectly, a score of 1 is given when the text cannot be seen and intermediate scores of 2 to 9 are given for progressively better clarity of the text. A minimum score of 8 is deemed a clear gel toothpaste.

In addition, a select set of samples were also evaluated for turbidity and transmittance according to the method reported in International Patent Publication No. WO2021002910A1, incorporated herein in its entirety, to correlate subjective assessments with analytical measurements. Turbidity for the tested toothpastes are tested on a Hach-2100Q portable turbidimeter. Turbidity is expressed on a scale from 0 to 1000 NTU, wherein 0 represents complete optical clarity. Transmittance for the toothpastes is tested on a Turbiscan LAB stability analyzer as percent of light transmitted (100% is optical clarity). The results are shown in Table 7 below. It should be noted that both turbidity and transmittance are dependent on the path length through the sample tested (turbidity and transmittance being linearly proportional to path length for homogenous samples) and while visual measurements were made on the dentifrice ribbon squeezed out of a toothpaste tube with an approximate thickness of 7-10 mm, the instruments used require filling a sample cube having a 24.8 mm path length with the tested toothpaste. As a result, values obtained for transmittance and turbidity are depressed compared to the values that would be achieved in practice, and should be considered for best correlation to visual impact.

TABLE 7
Turbidity & Transmittance Measurements of Toothpaste Gels
Formulas of Example 1 (only zinc)

| | A | B | C | D | H | J |
|---|---|---|---|---|---|---|
| Visual Clarity (10 = very | 10 | 3 | 4 | 8 | 8 | 1 |
| clear, l = very opaque) | | | | | | |
| Turbidity (0-1000NTU) | 83 | 357 | 323 | 138 | — | 998 |
| Transmittance (%) | 24 | 9 | 11 | 19 | — | 1.2 |

Formulas of Example 1
(both zinc and stannous)

| | N | O | P | Q |
|---|---|---|---|---|
| Visual Clarity (10 = very | 9 | 8 | 2 | 8 |
| clear, l = very opaque) | | | | |
| Turbidity (0-1000NTU) | — | 146 | 923 | — |
| Transmittance (%) | — | 22 | 3.8 | — |

The results show that Formulas A, D, H, N, O and Q, having the higher values for % of soluble metal (zinc, or zinc and stannous) relative to total metal have surprisingly high levels of clarity and transparency. In contrast, Formulas B, C, J and P have substantially lower clarity and transparency.

### Example 4

Refractive Index of Sylodent VP5 Silica

The refractive index of 4% Sylodent VP5 Silica in water/sorbitol solutions was determined using a Spectronic 21D Spectrophotometer at 589 nm wavelength (refractometer range=1.435-1.52). The % Transmittance of VP5 Silica solutions, and also two other commercially available high cleaning silicas was determined using a Shimadzu UV-1601PC Spectrophotometer, also at 589 nm wavelength. The results are shown in Table 8 below.

TABLE 8
Refractive Index and % Transmittance of Silica

| | | | | | % Transmittance | | | |
|---|---|---|---|---|---|---|---|---|
| | | | | Sorbitol/ | | | Other High | Other High |
| | % | Sorbitol | Water | Water | No | VP5 | Cleaning | Cleaning |
| | Sorbitol | Wt (g) | Wt (g) | R.I. | Silica | Silica | Silica #1 | Silica #2 |
| Sol'n | 50 | 100 | 100 | | | | | |
| ID | 60 | 120 | 80 | | | | | |
| | 70 | 140 | 60 | | | | | |
| A | 80 | 160 | 40 | — | 99.85 | 4.48 | 8.84 | 8.86 |
| B | 85 | 170 | 30 | 1.439 | 99.88 | 15.21 | 27.31 | 24.18 |
| C | 87 | 174 | 26 | 1.442 | 99.98 | 24.35 | 46.80 | 40.09 |
| D | 90 | 180 | 20 | 1.446 | 100.48 | 38.33 | 70.10 | 68.38 |
| E | 93 | 186 | 14 | 1.451 | 100.06 | 88.02 | 89.89 | 94.65 |
| F | 95 | 190 | 10 | 1.454 | 99.04 | 94.64 | 76.99 | 95.50 |
| | 97 | 194 | 6 | | | | | |
| | 100 | 200 | 0 | | | | | |

Note:
Spectronic 21D Spectrophotometer, 589 nm wavelength/used for refractive index Refractometer range = 1.435-1.52
Note:
Shimadzu UV-1601PC Spectrophotometer, 589 nm wavelength/used for % Transmittance

It has been discovered in accordance with the present invention that both the refractive index (RI) of the gel phase (humectants, water, surfactants and in some cases flavor) and the RI of silicas in the formulation should closely match. Sylodent VP5 Silica is unique in that it is one of a very few high cleaning silicas with a desirable RI that provides effective clarity with a metal-containing toothpaste, particularly where the metals are sufficiently solubilized as described herein. Thus, the formulations of the present disclosure utilizing tri sodium citrate and other materials to improve metal solubility provide transparent gels that also boost antibacterial performance.

1. A translucent oral care composition comprising:
a. One or more zinc ion source(s) and/or stannous ion source(s); wherein the zinc and/or stannous ion source(s) are in amounts effective to provide at least 28% soluble zinc and/or stannous as a fraction of the total zinc and/or stannous ion concentration in the composition;
b. An abrasive (e.g., silica), wherein the abrasive has a refractive index of approximately 1.45 as measured in a 4% silica, 90% sorbitol/water solution; and
c. An orally acceptable vehicle.

2. The oral care composition of claim 1, wherein the composition comprises one or more zinc ion sources, and wherein the one or more sources of zinc ion source(s) comprises a zinc salt selected from the group consisting of: zinc citrate, zinc oxide, zinc phosphate, zinc lactate, zinc sulfate, zinc silicate and combinations thereof.

3. The oral care composition of claim 1, wherein the composition comprises a source of zinc ion, and wherein the zinc ion source comprises zinc oxide.

4. The oral care composition of claim 1, wherein the composition comprises a source of zinc ion, and wherein the zinc ion source comprises zinc citrate.

5. The oral care composition of claim 1, wherein the composition comprises one or more sources of zinc ion, and wherein the one or more sources of zinc ion comprises zinc oxide and zinc citrate.

6. The oral care composition of claim 1, wherein the composition comprises a source of zinc ion, and wherein the zinc ion source comprises zinc phosphate.

7. The oral care composition of claim 1, wherein the composition comprises a stannous ion source.

8. The oral care composition of claim 7, wherein the stannous ion source is selected from the group consisting of: stannous fluoride, other stannous halides (e.g., stannous chloride dihydrate), stannous pyrophosphate, organic stannous carboxylate salts, stannous ethylene glyoxide, and combinations thereof.

9. The oral care composition of claim 7, wherein the stannous ion source is stannous fluoride.

10. The oral care composition of claim 1, wherein the composition comprises a silica abrasive having a N₂ BET surface area of less than 50 m²/g and an Einlehner hardness of from 4 to 11; for example wherein the silica abrasive (e.g., Sylodent VP5) has the following physical properties:

| | |
|---|---|
| N₂ BET surface area (m²/g) | <50 |
| Oil absorption (cc/100 g) | 80-100 |
| Mean particle size (μm) | 9-13 |
| d10 (μm) | 2.74 |
| Brass Einlehner hardness | 6-9 |

11. (canceled)

12. The oral care composition of claim 1, wherein the silica abrasive has a refractive index of approximately 1.45 as measured in a 4% silica, 90% sorbitol/water solution.

13. The oral care composition of claim 1 comprising an amino acid source, wherein the amino acid source comprises a basic amino acid; for example wherein the amino acid source comprises an amino acid selected from the group consisting of arginine, lysine, glycine and combinations thereof for example wherein the amino acid source comprises arginine.

14. (canceled)

15. (canceled)

16. The oral care composition of claim 1, wherein the composition comprises:
a. Zinc oxide and/or zinc citrate;
b. sodium fluoride;
c. an abrasive (e.g., silica), wherein the abrasive has a refractive index of approximately 1.45 as measured in a 4% silica, 90% sorbitol/water solution;
d. wherein the amounts of zinc oxide and zinc citrate are effective to provide at least 28% soluble zinc ion concentration as a fraction of the total concentration in the composition;
e. an orally acceptable carrier; and
f. arginine.

17. The oral care composition of claim 1, wherein the composition comprises:
a. zinc phosphate
b. stannous fluoride;
c. An abrasive (e.g., silica), wherein the abrasive has a refractive index of approximately 1.45 as measured in a 4% silica, 90% sorbitol/water solution;
d. an orally acceptable carrier;
wherein the amount of zinc phosphate and stannous fluoride is effective to provide at least 28% soluble metal ion concentration as a fraction of the total concentration in the composition; and
wherein the amount of water is more than 10% by wt. of the composition;
an orally acceptable carrier.

18. The oral care composition of claim 1, wherein the composition comprises sodium citrate; for example wherein the composition comprises trisodium citrate; for example wherein the composition comprises trisodium citrate in an amount from 2%-7% by wt. of the total composition.

19. (canceled)

20. (canceled)

21. The oral care composition of claim 1, wherein the amount of soluble metal ion(s) in the composition is from 28%-95%.

22. (canceled)

23. A method to improve oral health comprising applying an effective amount of the oral composition of claim 1 to the oral cavity of a subject in need thereof.

24. A method for producing a translucent oral care composition according to claim 1, wherein the method comprises combining one or more zinc ion source(s) and/or stannous ion source(s) in an orally acceptable carrier;
wherein the zinc and/or stannous ion source(s) are in amounts effective to provide at least 28% soluble zinc and/or stannous as a fraction of the total zinc and/or stannous ion concentration in the composition);
and combining with an abrasive, wherein the abrasive has a refractive index of approximately 1-2 as measured in a 4% silica, 90% sorbitol/water solution; and sodium citrate in an amount from 2%-7% by wt. of the total composition.

25. A method for increasing:
a) antibacterial efficacy; and/or
b) optical transmission
of an aqueous oral care composition, said composition comprising one or more zinc ion source(s) and/or stannous ion source(s), and an abrasive (e.g., silica), wherein the abrasive has a refractive index of approximately 1.45 as measured in a 4% silica, 90% sorbitol/water solution;
the method comprising formulating the composition to include a zinc ion and/or stannous ion solubilizing agent; e.g. wherein the solubilizing agent comprises citrate ion; e.g. wherein the solubilizing agent comprises trisodium citrate.

26. The method according to claim 25, wherein the aqueous oral care composition is a composition according to claim 1.
